(19) United States
(12) Patent Application Publication
(10) Pub. No.: US 2020/0129678 A1
(43) Pub. Date: Apr. 30, 2020

(54) VACUUM DRIVEN SUCTION AND IRRIGATION SYSTEM

(71) Applicant: Covidien LP, Mansfield, MA (US)

(72) Inventors: Jaroslaw Malkowski, Trumbull, CT (US); Kenneth Horton, South Glastonbury, CT (US); Lau Ow, Shatin (HK)

(21) Appl. No.: 16/589,651

(22) Filed: Oct. 1, 2019

Related U.S. Application Data
(60) Provisional application No. 62/750,301, filed on Oct. 25, 2018.

(51) Int. Cl. A61M 1/00

(52) U.S. Cl. CPC A61M 1/0058; A61M 1/0072; A61M 1/0084

(57) ABSTRACT

A vacuum assisted suction and irrigation system includes a suction and irrigation wand, an irrigation fluid supply, a vacuum source, and a fluid pump. The vacuum source is connected to a suction valve of the suction and irrigation wand to provide suction within the suction and irrigation wand. The irrigation fluid supply is connected to the suction and irrigation wand via the fluid pump to supply pressurized irrigation fluid to the suction and irrigation wand. The vacuum source is connected to the fluid pump to pressurize the irrigation fluid being delivered to the suction and irrigation wand.

### CROSS-REFERENCE TO RELATED APPLICATIONS

This application claims the benefit of and priority to U.S. Provisional Patent Application Ser. No. 62/750,301 filed Oct. 25, 2018, the entire disclosure of which is incorporated by reference herein.

### BACKGROUND

### 1. Technical Description

The present disclosure is directed to a suction and irrigation system and, more particularly, to a vacuum assisted suction and irrigation system.

### 2. Background of Related Art

Suction and irrigation systems that include a surgical instrument to provide both suction and irrigation to a surgical site are well known. Typically, such systems include a source of vacuum that is coupled to the surgical instrument to provide suction and an electric, e.g., battery powered, pump to deliver irrigation fluid to the surgical instrument. Thus, two types of power sources are required to use the surgical instrument.

A continuing need exists in the surgical arts for a suction and irrigation system that can operate using a single energy source, i.e., vacuum, that is already available in an operating room.

### SUMMARY

One aspect of the present disclosure is directed to a vacuum assisted suction and irrigation system that includes a suction and irrigation wand, an irrigation fluid supply, a vacuum source, and a fluid pump. The suction and irrigation wand includes a proximal body portion supporting a suction valve and an irrigation valve and a distal body portion defining a fluid channel. The irrigation fluid supply is connected to the irrigation valve of the suction and irrigation wand. The irrigation valve is actuable to deliver irrigation fluid to the fluid channel of the suction and irrigation wand. The vacuum source is connected to the suction valve of the suction and irrigation wand. The suction valve is actuable to draw a vacuum within the fluid channel. The fluid pump is positioned to deliver fluid to the suction and irrigation wand. The vacuum source is connected to the fluid pump to pressurize the irrigation fluid.

In embodiments, the fluid pump is a diaphragm type pump.

In some embodiments, the fluid pump includes a first pump chamber and a second pump chamber. The first pump chamber is divided into a first vacuum cavity and a first fluid cavity by a first diaphragm and the second pump chamber is divided into a second vacuum cavity and a second fluid cavity by a second diaphragm.

In certain embodiments, the first diaphragm supports a first piston and the second diaphragm supports a second piston.

In embodiments, the first piston is coupled to the second piston by a piston shaft.

In some embodiments, the fluid pump includes a vacuum chamber and a vent channel, wherein the vacuum chamber communicates with the vacuum source and the vent channel communicates with atmosphere.

In certain embodiments, the fluid pump includes a vent valve assembly, a first bore communicating the first vacuum cavity with the vent channel, and a second bore communicating the second vacuum cavity with the vent channel.

In embodiments, the vent valve assembly includes a first valve member and a second valve member and is movable between a first position in which the first valve member seals the first bore and the second bore is unsealed, and a second position in which the second seal member seals the second bore and the first bore is unsealed.

In some embodiments, the fluid pump includes a vacuum valve assembly, a third bore communicating the first vacuum cavity with the vacuum chamber, and a fourth bore communicating the second vacuum cavity with the vacuum chamber.

In certain embodiments, the vacuum valve assembly includes a third valve member and a fourth valve member and is movable between a first position in which the third valve member seals the third bore and the fourth bore is unsealed, and a second position in which the fourth seal member seals the fourth bore and the third bore is unsealed.

In embodiments, the first valve member is coupled to the second valve member by a second piston shaft and the third valve member is coupled to the fourth valve member by a third piston shaft.

In some embodiments, the irrigation fluid supply includes a compressible pouch.

In certain embodiments, the fluid pump includes a base member and a compression member having a first end pivotally coupled to the base member.

In embodiments, the compressible pouch is positioned between the base member and the compression member.

In some embodiments, the fluid pump includes a bellows that is attached to the base member and the compression member. The bellows is movable between a contracted position and an expanded position to control the pressure of irrigation fluid within the compressible pouch.

### BRIEF DESCRIPTION OF THE DRAWINGS

Various embodiments of the presently disclosed vacuum assisted suction and irrigation systems are described herein below with reference to the drawings, wherein:

FIG. 1 is a side perspective, schematic view of one exemplary embodiment of the presently disclosed vacuum assisted suction and irrigation system;

FIG. 2 is a side perspective view of a fluid pump of the vacuum assisted suction and irrigation system shown in FIG. 1;

FIG. 3 is an exploded perspective view of the fluid pump of FIG. 2;

FIG. 4 is an enlarged view of the indicated area of detail shown in FIG. 3;

FIG. 5 is a side perspective view of the valve assembly of the fluid pump shown in FIG. 2;

FIG. 6 is a cross-sectional view taken along section line 6-6 of FIG. 2;

FIG. 7 is a cross-sectional view taken along section line 7-7 of FIG. 2;

FIG. 8 is a side cross-sectional view of the of the fluid pump shown in FIG. 2 as fluid is pumped from a first pump chamber of the fluid pump;

FIG. 9 is a cross-sectional view taken along section line 9-9 of FIG. 8;

FIG. 10 is a side cross-sectional view of the of the fluid pump shown in FIG. 2 as fluid is pumped from a second pump chamber of the fluid pump;

FIG. 11 is a cross-sectional view taken along section line 9-9 of FIG. 8;

FIG. 12 a side perspective, schematic view of another exemplary embodiment of the presently disclosed vacuum assisted suction and irrigation system; and

FIG. 13 is a side cross-sectional view of the fluid pump of the vacuum assisted suction and irrigation system shown in FIG. 12.

### DETAILED DESCRIPTION OF EMBODIMENTS

The presently disclosed vacuum assisted suction and irrigation systems will now be described in detail with reference to the drawings in which like reference numerals designate identical or corresponding elements in each of the several views. However, it is to be understood that the disclosed embodiments are merely exemplary of the disclosure and may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

In this description, the term “proximal” is used generally to refer to that portion of the device that is closer to a clinician, while the term “distal” is used generally to refer to that portion of the device that is farther from the clinician. In addition, the term “clinician” is used generally to refer to medical personnel including doctors, nurses, and support personnel.

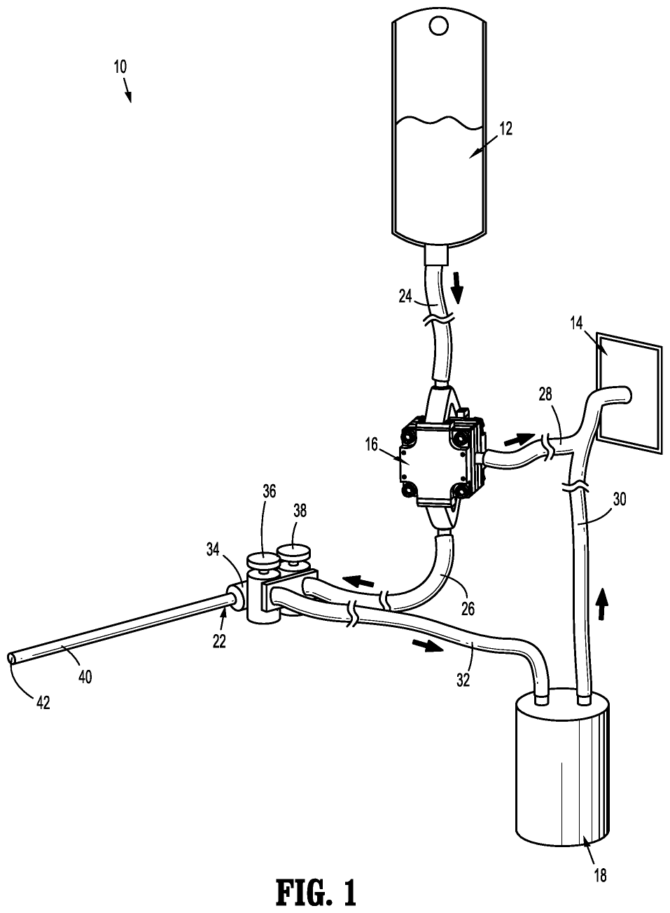

An exemplary embodiment of the presently disclosed vacuum assisted suction and irrigation system is shown generally in FIG. 1 as system 10. The system 10 includes an irrigation fluid supply 12, a vacuum source 14, a fluid pump 16, a suction container 18, and a suction and irrigation wand 22. The components of the device 10 are fluidly coupled to each other with fluid conduits 24-32 as described in further detail below.

The suction and irrigation wand 22 includes a proximal body portion 34 that supports a suction valve 36 and an irrigation valve 38, and a distal body portion 40 that defines a fluid channel 42 for receiving or dispensing fluid depending on which valve of the suction valve 36 and irrigation valve 38 is actuated. In embodiments, the proximal body portion 34 of the suction and irrigation wand 22 is connected to the irrigation fluid supply 12 by the fluid conduits 24 and 26 such that upon actuation of the irrigation valve 38, irrigation fluid is delivered to the fluid channel 42 of the distal body portion 40 of the suction and irrigation wand 22. Similarly, the proximal body portion 34 of the suction and irrigation wand 22 is connected to the vacuum source 14 by the fluid conduits 30 and 32 via the suction container 18 such that upon actuation of the suction valve 36, a vacuum is created in the fluid channel 42 of the distal body portion 40 of the suction and irrigation wand 22 to draw fluid into the fluid channel 42.

In embodiments, the suction and irrigation wand 22 communicates with the suction container 18 via the fluid conduit 32 and the suction container 18 communicates with the vacuum source 14 via the fluid conduit 30. As such, a vacuum is maintained within the suction container 18 by the vacuum source 14. Thus, when the suction valve 36 is actuated, the fluid channel 42 in the suction and irrigation wand 22 is communicated with the suction container 20 to create a vacuum within the fluid channel 42. All materials drawn into the channel 42 of the suction and irrigation wand 22, such as blood, tissue, saline, etc., are delivered to and collected in the suction container 20.

In some embodiments, the irrigation supply 12 is coupled to the fluid pump 16 by fluid conduit 24 and the fluid pump 16 is coupled to the suction and irrigation wand 22 by the fluid conduit 26 such that when the irrigation valve 38 is actuated, fluid from the irrigation fluid supply 12 is delivered to the suction and irrigation wand 22 via the fluid pump 16. In some embodiments, as described in further detail below, the irrigation fluid supply 12 can be connected directly to the suction and irrigation wand 22 and alternative means can be provided to pressurize the irrigation fluid supply 12.

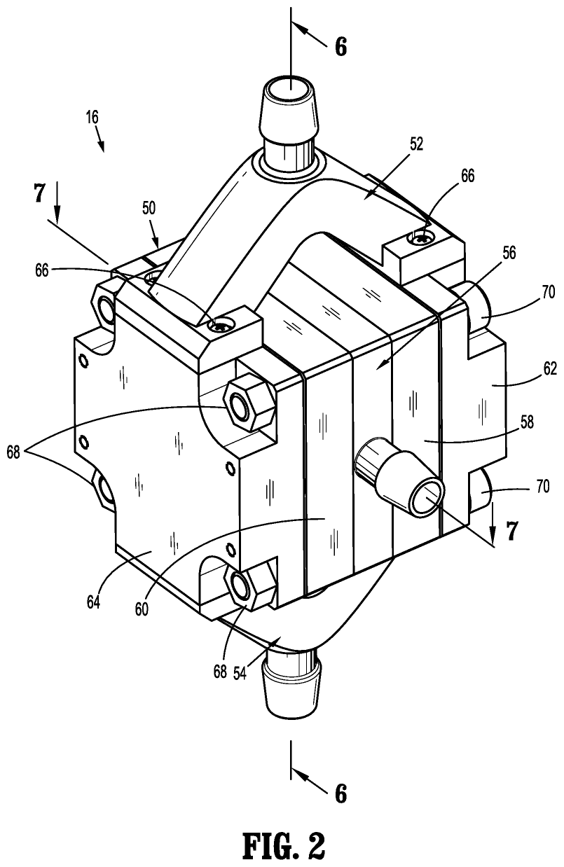

FIGS. 2-7 illustrate an exemplary embodiment of the fluid pump 16. Referring initially to FIG. 2, in embodiments, the fluid pump 16 is a diaphragm type pump and includes a housing 50, a fluid inlet manifold 52, and a fluid outlet manifold 54. The housing 50 includes a central housing portion 56, a first pump chamber body portion 58, a second pump chamber body portion 60, a first pump chamber cover 62, and a second pump chamber cover 64. The fluid inlet manifold 52 is coupled to the irrigation fluid supply 12 via the fluid conduit 24 (FIG. 1) and the fluid outlet manifold 54 is coupled to the suction and irrigation wand 22 via fluid conduit 26. In embodiments, the fluid inlet and outlet manifolds 52, 54 can be secured to the housing 50 using screws 66 or the like. Similarly, the different components of the housing 50 can be secured together using nuts 68 and bolts 70 or the like.

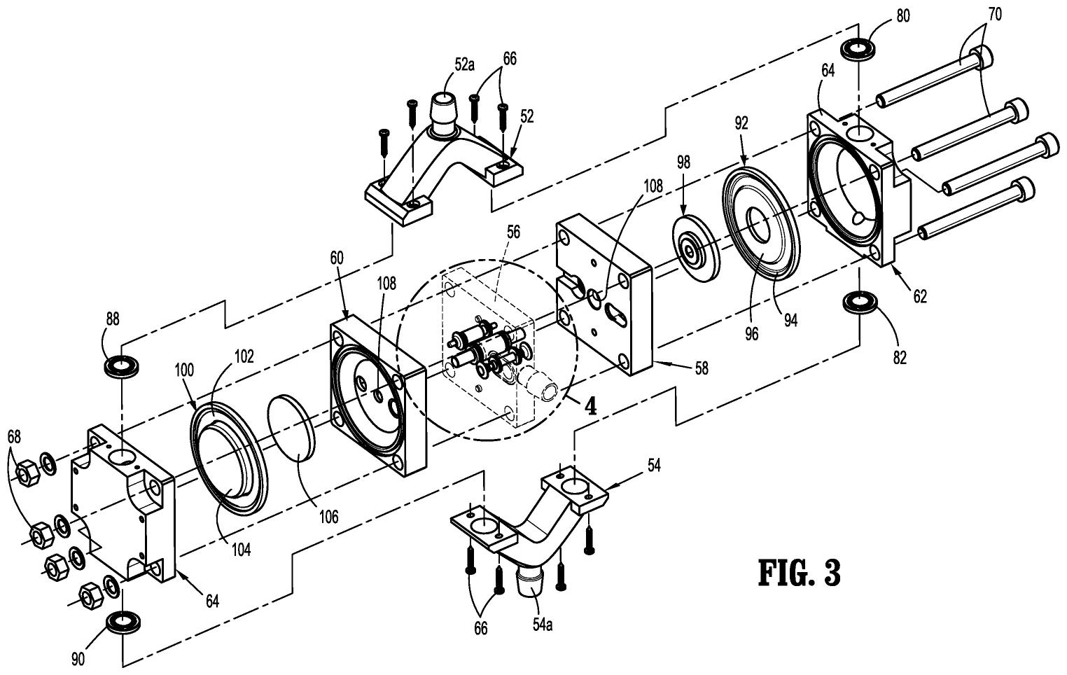

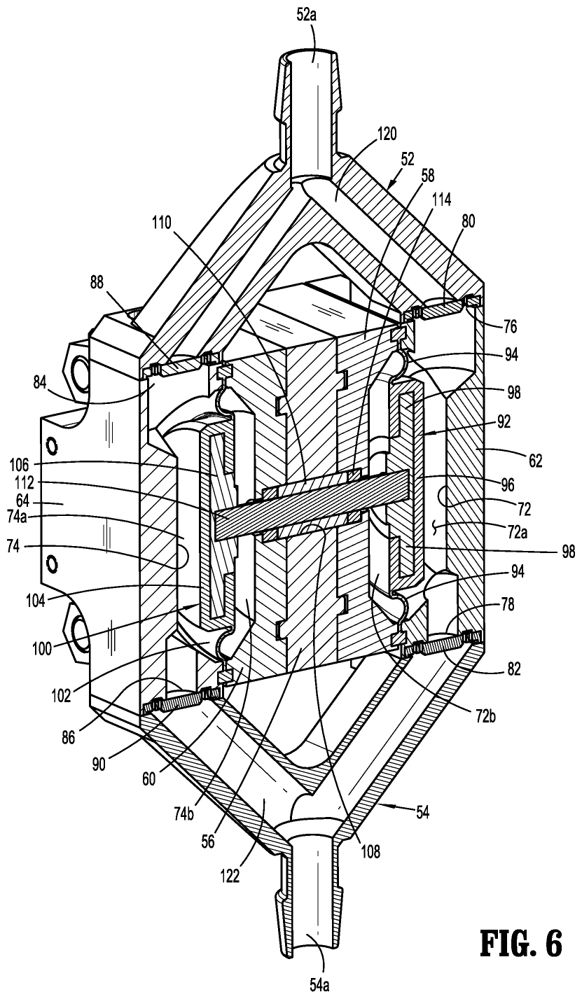

Referring also to FIGS. 3-7, the fluid pump 16 includes two pump chambers 72, 74 (FIG. 6). The first pump chamber 72 is defined between the first pump chamber body portion 58 and the first pump chamber cover portion 62. The second pump chamber 74 is defined between the second pump chamber body portion 60 and the second pump chamber cover portion 64. The first pump chamber 72 defines an inlet opening 76 (FIG. 6) and a fluid outlet opening 78 (FIG. 6). Each of the inlet and outlet openings 76, 78, is sealed by a check valve 80, 82, respectively. Similarly, the second pump chamber 74 defines an inlet opening 84 and a fluid outlet opening 86. Each of the inlet and outlet openings 84, 86 is sealed by a check valve 88, 90, respectively.

The first pump chamber 72 is divided into a fluid cavity 72a and a vacuum cavity 72b by a diaphragm 92. In embodiments, the diaphragm 92 includes an outer flexible portion 94 of reduced thickness and a central more rigid portion 96 that is configured to support a first piston 98. Similarly, the second pump chamber 74 is divided into a fluid cavity 74a and a vacuum cavity 74b by a diaphragm 100. In embodiments, the diaphragm 100 includes an outer flexible portion 102 of reduced thickness and a central more rigid portion 104 that is configured to support a second piston 106.

In embodiments, the central housing portion 56 and the body portions 58, 60 of the housing 50 define a bore 108 that that extends between the first and second pump chambers 72, 74. The bore 108 receives a bushing 110 and a piston shaft 112 that has a first end secured to the first piston 98 and a second end secured to the second piston 106. As such, movement of the first piston 98 within the first pump chamber 72 causes corresponding movement of the second piston 106 within the second pump chamber 74. A seal 114 is supported about the piston shaft 112 at each end of the bushing 110 to provide a seal about the piston shaft 112 at each end of the bore 108.

Referring briefly to FIG. 6, the inlet manifold 52 defines a bifurcated or Y-shaped fluid channel 120 having an inlet 52a that communicates with the inlet openings 76, 84 of the first and second pump chambers 72, 74, respectively. The outlet manifold 54 defines a Y-shaped fluid channel 122 that has an outlet 54a that communicates with the outlet openings 78, 86 of the first and second pump chambers 72, 74, respectively. The check valves 80, 82, 88 and 90 are configured to allow fluid to flow in one direction, i.e., into the first and second pump chambers 72, 74 from the inlet manifold 52 and out of the first and second pump chambers 72, 74 through the outlet manifold 54.

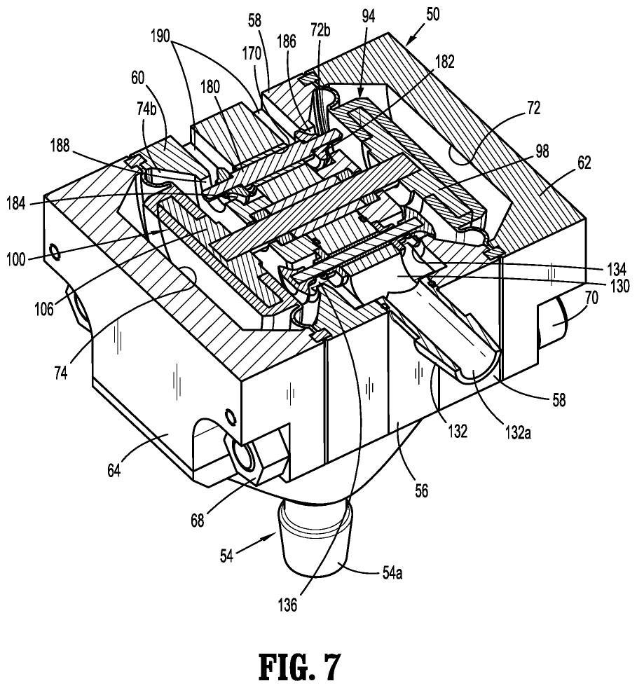

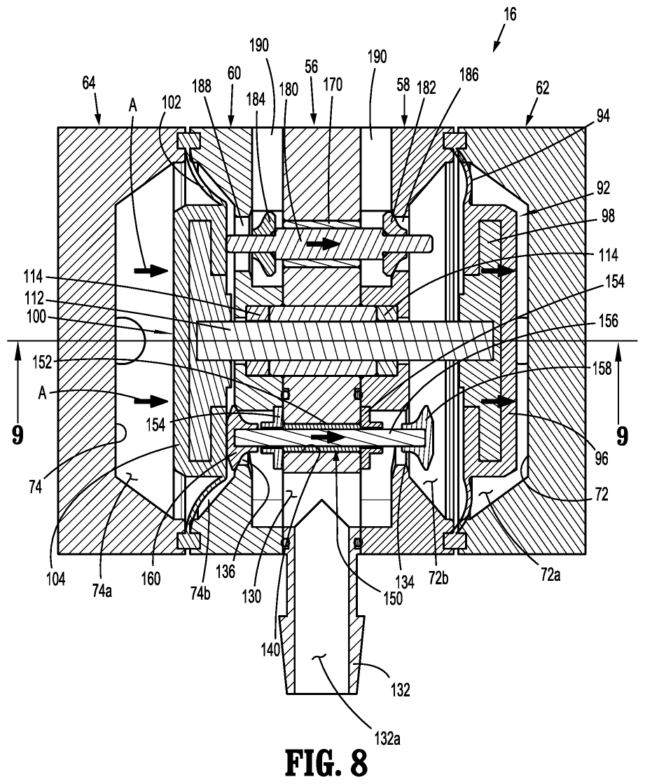

Referring again to FIGS. 3-7, the central housing portion 56 and the first and second pump chamber body portions 58, 60 of the housing 50 of the fluid pump 16 define a vacuum chamber 130 and a vacuum port 132 (FIG. 7). The vacuum port 132 defines a channel 132a that communicates with the vacuum chamber 130. The first pump chamber body portion 58 defines a bore 134 (FIG. 8) that communicates the vacuum chamber 130 with the vacuum cavity 72b of the first pump chamber 72. Similarly, the second pump chamber body portion 60 defines a bore 136 that communicates the vacuum chamber 130 with the vacuum cavity 74b of the second pump chamber 74. The bores 134, 136 are positioned to facilitate application of a vacuum to the vacuum cavities 72b, 74b of the first and second pump chambers 72, 74 of the fluid pump 16 to control movement of the diaphragms 94, 100 within the first and second pump chambers 72, 74 as described in more detail below.

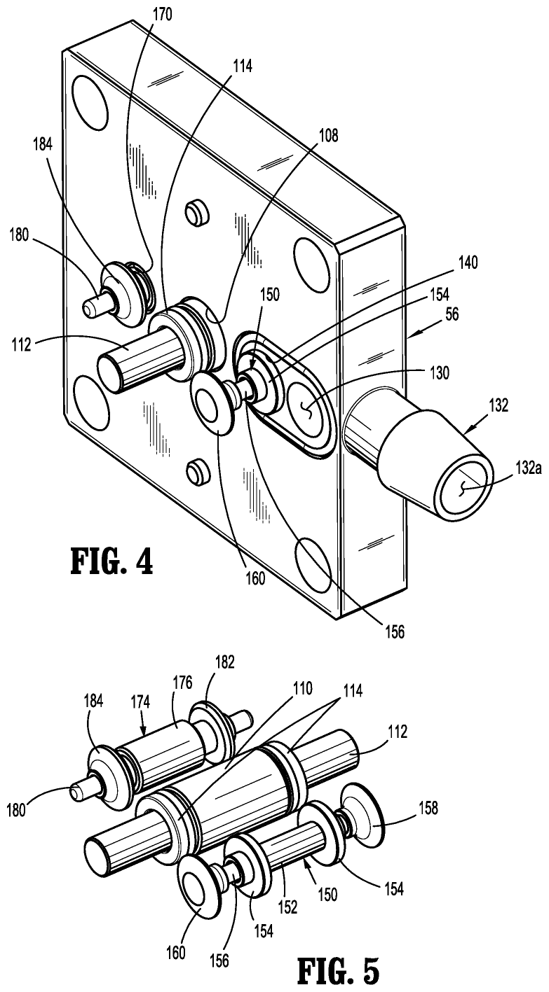

The central housing portion 56 (FIG. 4) of the housing 50 of the fluid pump 16 defines a bore 140 that receives a vacuum valve assembly 150. The vacuum valve assembly 150 includes a bushing 152, first and second seal members 154, a valve shaft 156, and valve members 158, 160 that are positioned to alternately seal the bores 134, 136 of the first and second pump chamber body portions 58, 60 (FIG. 5). The valve shaft 156 connects the valve member 158 to the valve member 160 and is movable between a first position in which the valve member 158 seals the bore 134 of the first pump chamber body portion 58 while the bore 136 of the second pump chamber body portion 60 remains unsealed and a second position in which the valve member 160 seals the bore 136 of the second pump chamber body portion 60 while the bore 134 of the first pump chamber body portion 58 remains unsealed. The first and second seal members 154 provide a seal about the valve shaft 156 at each end of the bore 140 of the central housing portion 56 to prevent fluid leakage between the first and second pump chambers 72, 74.

The central housing portion 56 of the housing 50 of the fluid pump 16 also defines a bore 170 (FIG. 4) that receives a vent valve assembly 174. The first and second pump chamber body portions 58, 60 define bores 186, 188. The vent valve assembly 174 includes a bushing 176, a valve shaft 180, and valve members 182, 184 that are positioned to alternately seal the bores 186, 188. The bores 186, 188 connect the vacuum cavities 72b, 74b of the first and second pump chambers 72, 74 with vent channels 190 (FIG. 7) formed in the first and second pump chamber body portions 58, 60. The valve shaft 180 connects the valve member 182 to the valve member 184 and is movable within the bushing 176 between a first position in which the valve member 182 seals the bore 186 of the first pump chamber body portion 58 while the bore 188 of the second pump chamber body portion 60 remains unsealed and a second position in which the valve member 184 seals the bore 188 of the second pump chamber body portion 60 while the bore 186 of the first pump chamber body portion 58 remains unsealed.

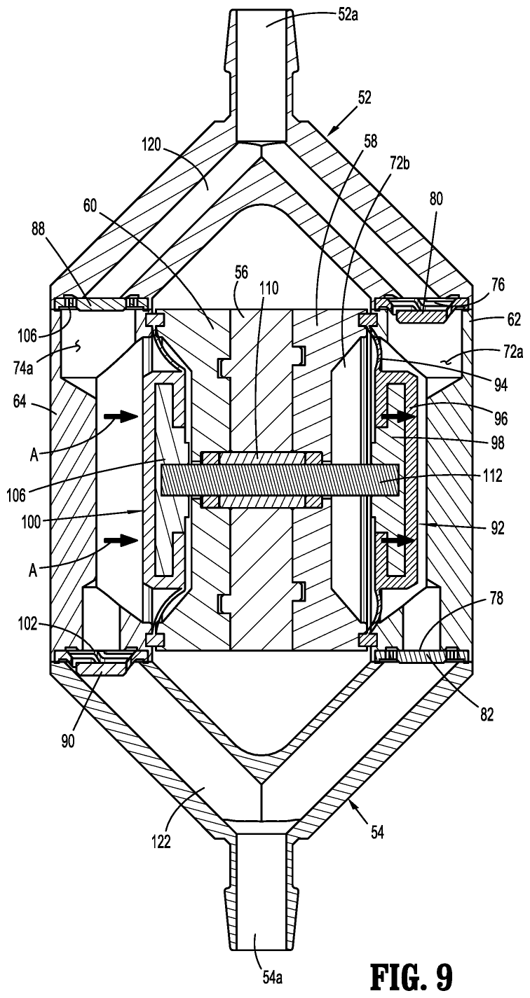

Referring to FIGS. 8 and 9, the valve shaft 180 of the vent valve assembly 174 and the valve member 160 of the vacuum valve assembly 150 are positioned to be engaged by the diaphragm 104 within the second pump chamber 74 when the diaphragm 104 moves in the direction indicated by arrows “A” to a position to collapse the vacuum cavity 74b of the second pump chamber 74. As the diaphragm 104 moves to the position shown in FIG. 8 in the direction of arrow “A”, the valve member 184 of the vent valve assembly 174 moves to a position to unseal the bore 188 connecting the vacuum cavity 74b of the second pump chamber 74 to the vent channels 190 and the valve member 182 moves to a position to seal the bore 186 connecting the vacuum cavity 72b of the first pump chamber 72 to the vent channels 190. Similarly, as the diaphragm 104 moves to the position shown in FIG. 8, the valve shaft 156 of the vacuum valve assembly 150 moves in the direction of arrows “A” to move the valve member 160 of the vacuum valve assembly 150 to a position to seal the bore 136 connecting the vacuum chamber 130 to the vacuum cavity 74b and the valve member 158 moves to a position to unseal the bore 134 connecting the vacuum chamber 130 to the vacuum cavity 72b of the first pump chamber 72. As the diaphragm 100 moves to the position in which the vacuum chamber 74b is collapsed, the fluid cavity 74a of the second pump cavity 74 is expanded to draw fluid into the fluid cavity 74a through the check valve 88. As the diaphragm 94 in the first pump cavity 72 moves in the direction indicated by arrows “A”, irrigation fluid is forced from the fluid cavity 72a of the first pump cavity 72 through the check valve 82 and into the fluid outlet manifold 54.

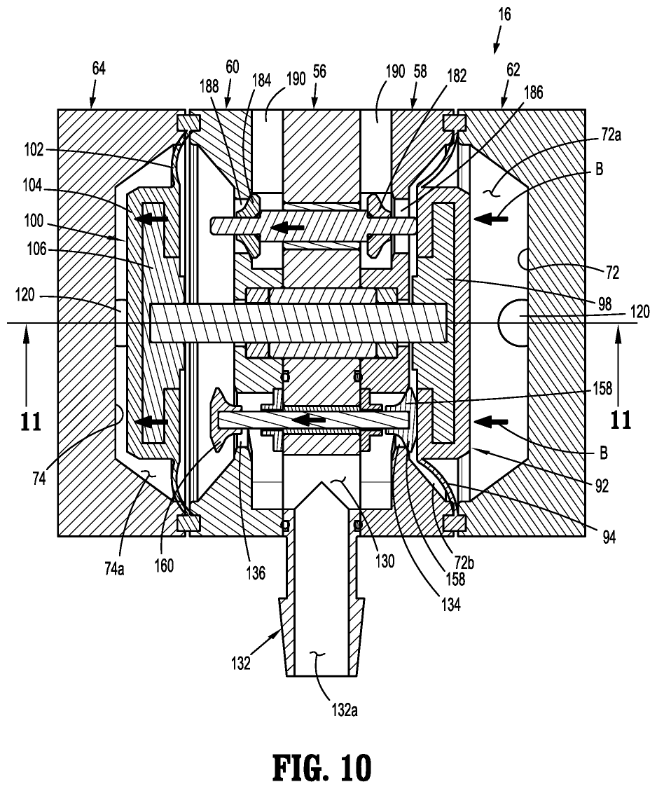

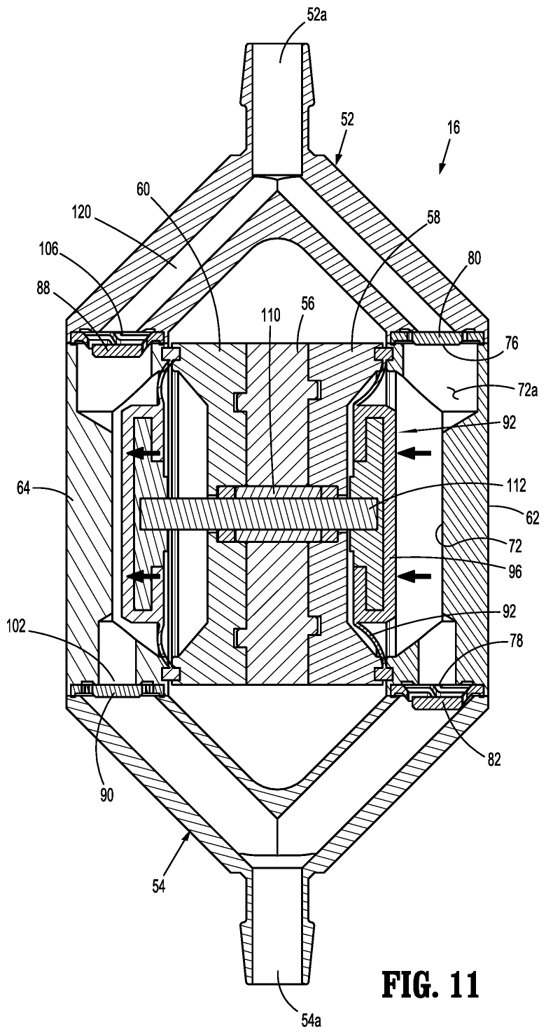

Referring also to FIGS. 10 and 11, when the valve member 158 (FIG. 8) unseals the bore 134 and the valve member 184 unseals the bore 188, a vacuum is drawn in the vacuum cavity 72b of the first pump chamber 72 and the vacuum cavity 74b of the second pump chamber 74 is vented. This difference in pressure in the vacuum cavities 72b, 74b of the first and second pump cavities 72, 74, respectively, causes the diaphragm 92 in the first pump cavity 72 to move in the direction of arrows “B” in FIGS. 10 and 11 to collapse the vacuum cavity 72b of the first pump chamber 72. As the vacuum cavity 72b collapses, the fluid cavity 72a of the first pump chamber 72 expands to draw irrigation fluid into the fluid cavity 72a through the first check valve 80 (FIG. 11). As the diaphragm 94 moves in the direction indicated by arrows “B”, the piston shaft 112 causes corresponding movement of the diaphragm 100 in the second pump chamber 74 to force irrigation fluid from the fluid cavity 74a of the second pump chamber 74 through the check valve 90 (FIG. 11) and to expand the vacuum cavity 74b of the second pump chamber 74.

As discussed above in regard to diaphragm 104, the diaphragm 94 is positioned to engage the valve shaft 180 of the vent valve assembly 174 and the valve member 158 of the vacuum valve assembly 150 when the diaphragm 94 moves in the direction indicated by arrows “B” to unseal the bore 130 connecting the vacuum chamber 74b of the second pump chamber 74 to the vacuum chamber 130 and to unseal the bore 186 connecting the vacuum cavity 72b of the first pump chamber 72 to vent. Once again, this difference in pressure in the vacuum cavities 72b, 74b of the first and second pump chambers 72, 74 causes the diaphragm 94 to change direction to movement in the direction of arrow “A” (FIG. 8). The diaphragms 94 and 100 will continue to operate in this manner to provide a pressurized supply of irrigation fluid to the irrigation and suction wand 22 (FIG. 1).

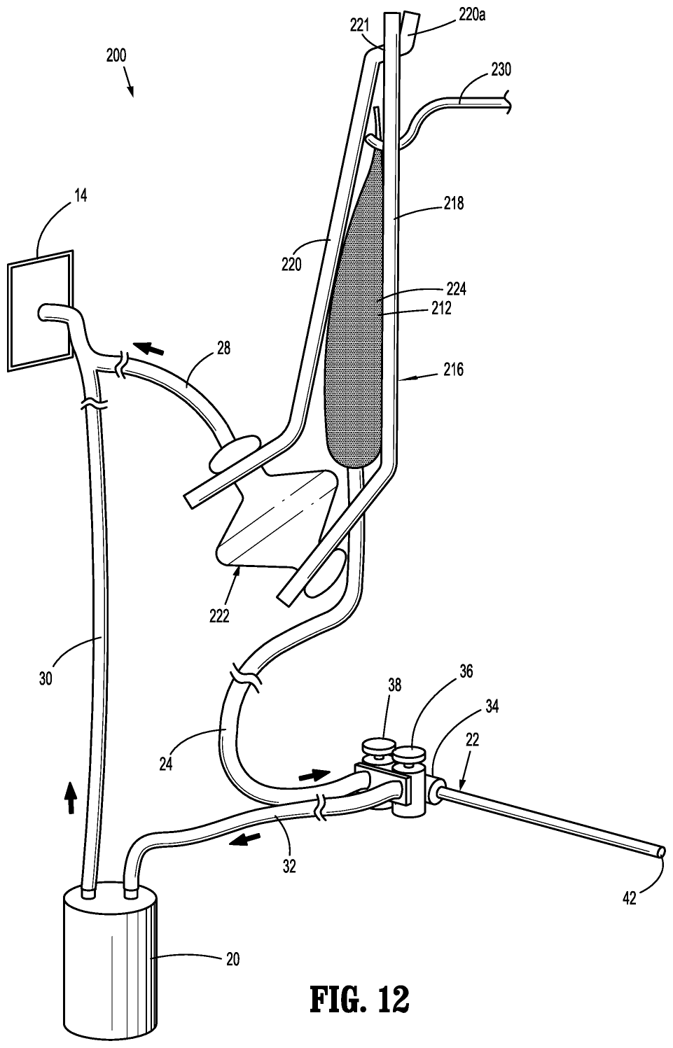

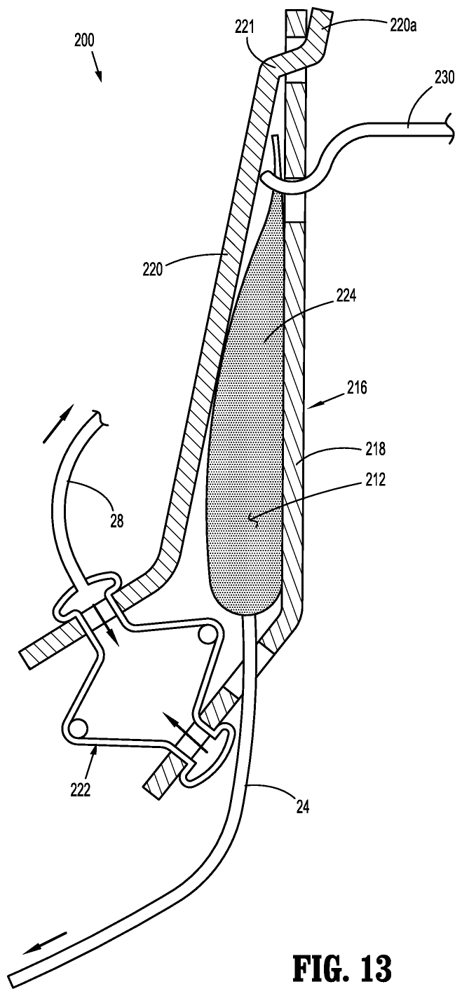

Referring to FIGS. 12 and 13, it is envisioned that a variety of different types of pumps that are driven by vacuum may be used in place of the fluid pump 16. For example, in the suction and irrigation system 200, a fluid pump 216 includes a base member 218, a compression member 220, and a bellows 222. The compression member 220 has a first end 220a that is pivotally supported on the base member 218 by a pivot member 221 which may have a variety of configurations. The irrigation fluid supply 212 includes a compressible pouch 224 that defines a receptacle that receives irrigation fluid. The compressible pouch 224 is supported between the base member 218 and the compression member 220. In embodiments, the compressible pouch 224 is supported on a hook member 230 that also supports the base member 218. Alternately, the compressible pouch 224 can be supported on the base member 218 using a hook or other type of coupling member.

The bellows 222 is secured to ends of the base member 218 and the compression member 220 opposite the pivot member 221. The bellows 222 is movable between contracted and expanded configurations to pivot the compression member 220 in relation to the base member 218 to pressurize the irrigation fluid within the compressible pouch 224. In embodiments, the bellows 222 is connected to the vacuum source 14 by a fluid conduit 28 to control expansion and contraction of the bellows 222. In embodiments, the pressure within the bellows 222 is controlled with the vacuum source 12 to maintain the pressure of the irrigation fluid within the compressible pouch 224 constant.

The vacuum assisted suction and irrigation systems 10 and 200 operate in substantially similar manners. More specifically, a clinician grasps the proximal body portion 34 of the suction and irrigation wand 22 and manipulates the wand to position a distal end of the distal body portion 40 adjacent a surgical site (not shown). Although not shown, this may include inserting the distal body portion 40 of the suction and irrigation wand 22 through a cannula assembly to access the surgical site. If the clinician requires suction at the surgical site, e.g., to remove blood or debris, the clinician can actuate the suction valve 36 to draw material from the surgical site into the channel 42 of the suction and irrigation wand 22. All material withdrawn into the suction and irrigation wand 22 is delivered to the suction container 20 via the fluid conduit 32. If the clinician requires irrigation fluid, e.g., to improve visualization of the surgical site, the clinician can actuate the irrigation valve 38 to deliver fluid to the channel 42 of the distal body portion 40 of the suction and irrigation wand 22. In both systems, vacuum from the vacuum source 14 assists in pressurizing the irrigation fluid to supply the irrigation fluid to the suction and irrigation wand 22.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. A vacuum assisted suction and irrigation system comprising:
a suction and irrigation wand including a proximal body portion supporting a suction valve and an irrigation valve and a distal body portion defining a fluid channel;
an irrigation fluid supply connected to the irrigation valve of the suction and irrigation wand, the irrigation valve being actuable to deliver irrigation fluid to the fluid channel of the suction and irrigation wand;
a vacuum source connected to the suction valve of the suction and irrigation wand, the suction valve being actuable to draw a vacuum within the fluid channel; and
a fluid pump configured to deliver irrigation fluid to the suction and irrigation wand, the vacuum source connected to the fluid pump to pressurize the irrigation fluid being delivered to the suction and irrigation wand.

2. The suction and irrigation system of claim 1, wherein the fluid pump is a diaphragm type pump.

3. The suction and irrigation system of claim 2, wherein the fluid pump includes a first pump chamber and a second pump chamber, the first pump chamber being divided into first vacuum cavity and a first fluid cavity by a first diaphragm and the second pump chamber being divided into second vacuum cavity and a second fluid cavity by a second diaphragm.

4. The suction and irrigation system of claim 3, wherein the first diaphragm supports a first piston and the second diaphragm supports a second piston.

5. The suction and irrigation system of claim 4, wherein the first piston is coupled to the second piston by a first piston shaft.

6. The suction and irrigation system of claim 5, wherein the fluid pump includes a vacuum chamber and a vent channel, the vacuum chamber communicating with the vacuum source and the vent channel communicating with atmosphere.

7. The suction and irrigation system of claim 6, wherein the fluid pump includes a vent valve assembly, a first bore communicating the first vacuum cavity with the vent channel, and a second bore communicating the second vacuum cavity with the vent channel.

8. The suction and irrigation system of claim 7, wherein the vent valve assembly includes a first valve member and a second valve member, the vent valve assembly being movable between a first position in which the first valve member seals the first bore and the second bore is unsealed, and a second position in which the second seal member seals the second bore and the first bore is unsealed.

9. The suction and irrigation system of claim 8, wherein the fluid pump includes a vacuum valve assembly, a third bore communicating the first vacuum cavity with the vacuum chamber, and a fourth bore communicating the second vacuum cavity with the vacuum chamber.

10. The suction and irrigation system of claim 9, wherein the vacuum valve assembly includes a third valve member and a fourth valve member, the vacuum valve assembly being movable between a first position in which the third valve member seals the third bore and the fourth bore is unsealed, and a second position in which the fourth seal member seals the fourth bore and the third bore is unsealed.

11. The suction and irrigation system of claim 8, wherein the first valve member is coupled to the second valve member by a second piston shaft and the third valve member is coupled to the fourth valve member by a third piston shaft.

12. The suction and irrigation system of claim 1, wherein the irrigation fluid supply includes a compressible pouch containing an irrigation fluid.

13. The suction and irrigation system of claim 12, wherein the fluid pump includes a base member and a compression member, the compression member having a first end pivotally coupled to the base member.

14. The suction and irrigation system of claim 13, wherein the compressible pouch is positioned between the base member and the compression member.

15. The suction and irrigation system of claim 14, wherein the fluid pump includes a bellows that is attached to the base member and the compression member, the bellows being movable between a contracted position and an expanded position to control the pressure of irrigation fluid within the compressible pouch.
